(19) United States
(12) Patent Application Publication
(10) Pub. No.: US 2017/0007434 A1
(43) Pub. Date: Jan. 12, 2017

(54) GI-ENDOCRINE DRUG DELIVERY DEVICE AND METHOD OF USE

(71) Applicant: Hamilton Scientific, Ltd., Roseland, NJ (US)

(72) Inventor: Michael Rothkopf, North Caldwell, NJ (US)

(73) Assignee: Hamilton Scientific, Ltd., Roseland, NJ (US)

(21) Appl. No.: 15/206,886

(22) Filed: Jul. 11, 2016

Related U.S. Application Data
(60) Provisional application No. 62/191,081, filed on Jul. 10, 2015.
(60) Provisional application No. 62/190,967, filed on Jul. 10, 2015.

(51) Int. Cl. A61F 5/00; A61M 5/172; A61M 5/142

(52) U.S. Cl. CPC A61F 5/0059; A61M 5/142; A61M 5/1723; A61F 2005/002; A61F 2005/0023; A61M 2005/14208; A61M 2210/1053; A61M 2230/005; A61M 2230/201; A61M 2205/3344; A61M 2230/06; A61M 2205/52; A61M 2205/3561; A61M 2205/3523

(57) ABSTRACT

Shown are a device, system and method that improve the diabetes care of a patient that has undergone gastric surgery. The device provides continuously or intermediate monitoring at various intervals before and after meals of several sensors to perceive, among other things, the blood glucose level, EKG/heart rate and volume of meals a patient has consumed. Readings are sent to a control unit that receives input from at least one of: a meal sensor, glucose monitor, and EKG sensor to control a drug delivery unit that doses a prescribed agent at a specific quantity and time based on the readings to provide proper diabetes care for the patient. Components of the system such as the meal sensor glucose monitor, EKG sensor, control unit and drug delivery device may be implanted subcutaneously in the patient or outside the patient and may communicate with each other either wirelessly or may be directly wired together.

### CROSS REFERENCE TO RELATED APPLICATION

This application claims benefit of U.S. Provisional Application Nos. 62/190,967 filed Jul. 10, 2015 entitled GI-ENDOCRINE DRUG DELIVERY DEVICE AND METHOD OF USE and 62/191,081 filed Jul. 10, 2015 entitled GI-ENDOCRINE DRUG DELIVERY DEVICE AND METHOD OF USE, the contents of which are incorporated by reference herein in its entirety for all purposes.

### BACKGROUND

Type 2 diabetes mellitus (T2DM) is a relentless disease affecting over 20 million people in the U.S. alone. The disease stems from the human body's inability to produce insulin or the human body's inability to recognize insulin. Because of the body's inability to produce or recognize insulin, people afflicted with T2DM do not properly utilize glucose for energy.

Traditionally, T2DM has been treated through diet, exercise and/or medication. Medical studies have indicated that this disease may be treated with bariatric surgeries, such as those that have been commonly used to reduce the size of the stomach in connection with the treatment of obesity. In light of these medical studies, metabolic surgical treatments for non-obese patients have been developed. Some surgical treatments typically entail a rearrangement of whole sections of the GI tract, for example, the transposition of one section of small bowel (e.g., ileum) to a more proximal section of the small bowel (e.g., jejunum).

The objective of such transposition surgeries is to modify the hormones which are produced in the proximal section of the GI tract with the intent of improving glucose homeostasis. It has been shown that such transposition surgeries can result in greater production of hormones in the proximal section of the GI tract that are associated with mediating diabetes. Such hormones may include Glucagon-like peptide-1 (GLP-1) and peptide-YY.

The drawback of these types of bariatric surgical techniques are that they are very traumatic and invasive surgeries involving anatomical reconstruction, oftentimes requiring incisions through multiple layers of tissue, general anesthesia and hours to perform. In addition, these types of surgeries are not easily reversed since it involves significantly modifying anatomical structures from the natural state of the body.

Diabetes resolution can be accomplished through a gastric bypass bariatric surgery which creates gastric restriction and increases the entero-endocrine system's secretion of incretin hormones, such as GLP-1. Less aggressive bariatric procedures, such as the gastric band, cannot achieve diabetes resolution with the same alacrity. However, combining gastric band surgery with the subcutaneous administration of an incretin-mimetic drug, augments the gastric band's capacity to induce diabetes resolution.

As an example, U.S. Pat. No. 8,710,002 to Rothkopf discloses compositions and methods for increasing diabetes resolution having undergone gastric restrictive surgery with the use of an active agent that produces an incretin-like effect in the patient. However, there is still a need to time the release of any active agent to coordinate the timing and amount of drug release needed for a particular patient over time.

Thus there still remains a need in the art to provide a drug delivery device that monitors the amount of glucose levels and releases a prescribed amount of active agent at a specific time as needed for the patient. There is also a need in furtherance of the above to monitor the volume of a patient's meal and frequency of meals. Furthermore, there is a need to coordinate monitoring heart rate in coordination with release of any active agent and utilization of such continuous readings.

### BRIEF SUMMARY OF THE INVENTION

The device, system, and method of the present invention solves the above problems and also provides solutions that allow monitoring of patient conditions that have undergone gastric surgery, and in addition release an active agent to enable physiologic control of drug delivery to avert emergency conditions in diabetics before they happen as well as provide vital information for the maintenance and diabetes care of the patient.

Depending on the embodiment, the invention may include various components for the drug delivery system, including but not limited to, a meal sensing apparatus, a continuous glucose monitor, an EKG monitor, a CPU system integrator/controller and a drug delivery system. Each component may be used individually or combined with each other in multiple fashions depending on the desired use for the patient.

Advantages of the present invention include monitoring the timing and volume of meals consumed by the patient in order to activate release of an agent for diabetes care. Embodiments of the invention also allows monitoring of blood glucose levels after eating in various intervals as well as continuous monitoring. The EKG monitor allows heart rate monitoring for further diabetes care and release of the active agent to control blood glucose levels.

The drug delivery device may or may not be implanted depending on the embodiment. An interface with a control unit and/or CPU to control release of the active agent may be implemented wirelessly or interfaced directly with any part of the above drug delivery system, for example.

The monitoring of meal intake, blood glucose levels and EKG may occur on a regular basis, which may be programmatically defined and may change during execution of the device's firmware. The device allows monitoring at least during a specified time period right before and after patient meals. In this embodiment, the timed monitoring allows specific response and diabetes care in relation to a patient's meal intake.

The foregoing objects are achieved and other features and advantages of the present invention will become more apparent in light of the following detailed description of exemplary embodiments thereof, as illustrated in the accompanying drawings.

### BRIEF DESCRIPTION OF THE DRAWINGS

FIG. 1 is a depiction of a device that may be utilized in accordance to the present invention;

FIG. 2 is a front view of the portal device in FIG. 1;

FIG. 3 is a cross-section view of the portal in FIG. 2 showing a transducer;

FIG. 4 is a perspective view of the transducer in FIG. 3;

FIG. 5 is a graph illustrating one embodiment of the device's data outputs for the device in FIG. 1;

FIG. 6 is a circuit diagram illustrating one embodiment of the device's glucose monitoring states for the device in FIG. 1;

FIG. 7 is a block diagram illustrating one embodiment of components for the device in FIG. 1;

FIG. 8A is a perspective view giving a depiction of one example of a drug delivery pump system for the device in FIG. 1; and

FIG. 8B is a side view of the drug delivery pump system in FIG. 8A.

### DETAILED DESCRIPTION

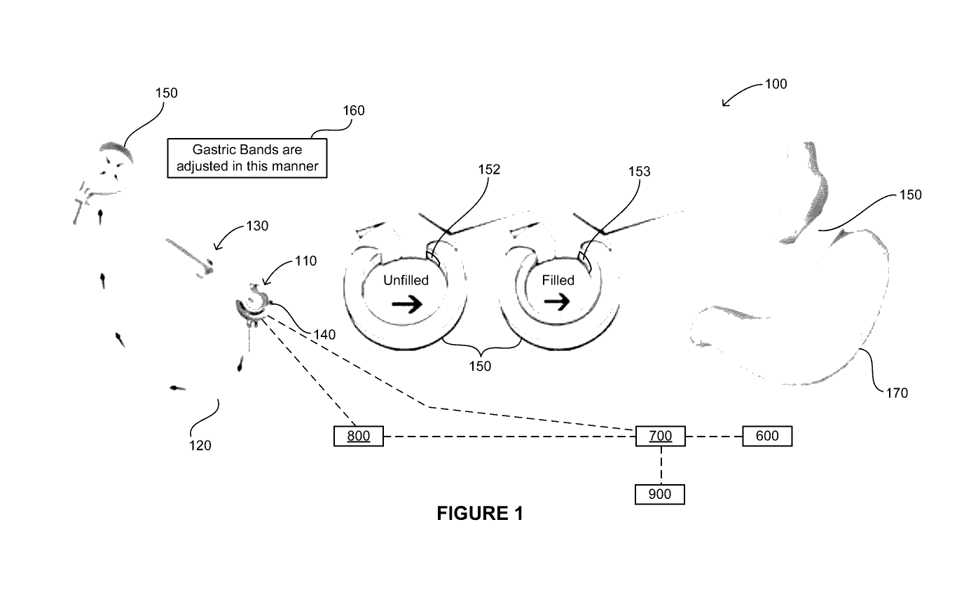

The invention will now be described in detail with reference to the accompanying drawings. As shown in FIG. 1, the present invention relates to a gastric drug delivery system having one or more of the following 5 components: a meal sensing apparatus 110, a continuous or intermittent glucose monitor 600, an EKG monitor 900, a CPU system integrator/controller 700 and a drug delivery system 800. Meal sensing apparatus (MSA) 110, depending on the embodiment, may contain a pressure transducer 310 that is embedded within the structure of the MSA having a subcutaneous port 320 and reservoir 340. Changes in the port pressure that occur with eating are recorded by the pressure transducer 310 and communicated to the CPU 700. Depending on the implementation, the reservoir 340 may be similar in shape and structure to that of an implanted infusion port in that it may contain a silicon core for port 320 and base material made of a hard shell, for example a medical grade plastic or metal.

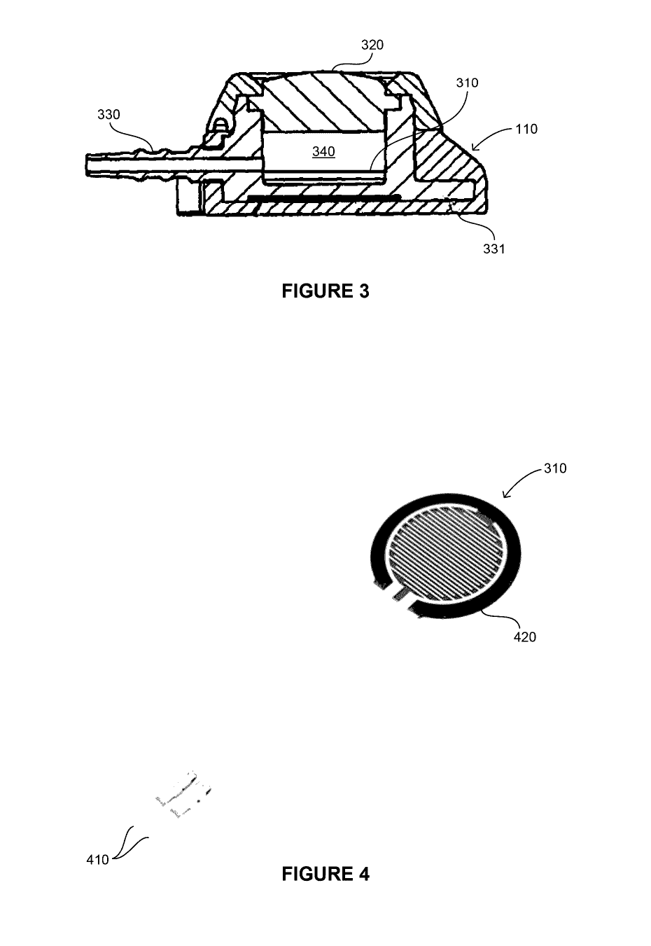

As shown in FIG. 3, the MSA is physically connected to a tube 120 of medical grade plastic or silicone via a sleeve 330 built into the base 331. The tube 120 carries fluid to a surgically implanted device such as a gastric band 150 or an intravenous catheter (not shown). There is an enclosed chamber or reservoir 340 within the port that permits instillation of a fluid.

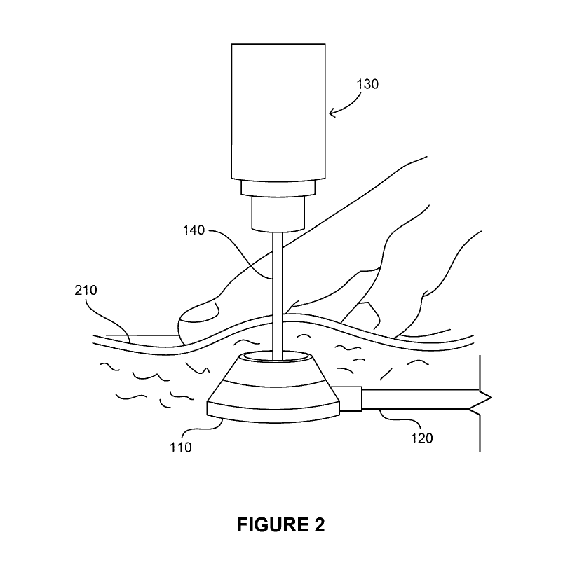

A syringe 130 with a needle 140 such as that having a Huber point, Whitaker point or other such needle point are inserted through the patient's skin 210 as illustrated in FIG. 2, and into the core 320 to access the reservoir 340 or chamber. The core 320 enables the needle to puncture the MSA port 110 numerous times without creating a permanent leak.

When the port is used in connection with a gastric band, the fluid in the chamber is connected to a bladder in the band ring. When fluid is inserted into the port, it inflates the bladder ring which puts pressure on the exterior gastric surface. This constricts the passage of food and produces a sense of fullness by the patient, resulting in weight loss.

The port chamber or reservoir 340, catheter 120, and band bladder deflated 152 or inflated 153 is a closed, confluent circuit. Changes in the chamber fluid pressure result in corresponding changes in the bladder fluid pressure. This permits the bladder to be filled or unfilled based on the patient's needs.

But the band bladder (152,153) can also act as a pressure sensor at the level of the gastric surface. As food passes through the upper stomach it causes a momentary deflection of the band bladder, changing the fluid pressure.

In the present invention, the port also contains a thin film pressure transducer 310 as shown in FIG. 3 and FIG. 4. This transducer 310 permits the port to act as a pressure sensor. The transducer 310 is able to sense and record the fluid pressure within the chamber/catheter/bladder circuit. The pressure increases as fluid is added to the circuit. It decreases as fluid is removed from the circuit. The pressure also increases and decreases as food pass through the gastric lumen beneath the banded area of the stomach. The passage of food impacts the gastric wall slightly, temporarily changing the band bladder dimensions. Since the fluid in the bladder is part of a closed volume, this change in bladder conformation results in a temporary change in pressure within the chamber 340.

As shown in FIG. 4, the pressure transducer 310 may be a film 420 within the port reservoir 340 and can sense and record pressure change in the reservoir 340 as a series of numerical values or a waveform. Leads 410 may be connected to a control unit 700 either wirelessly or directly to send data regarding the pressure changes.

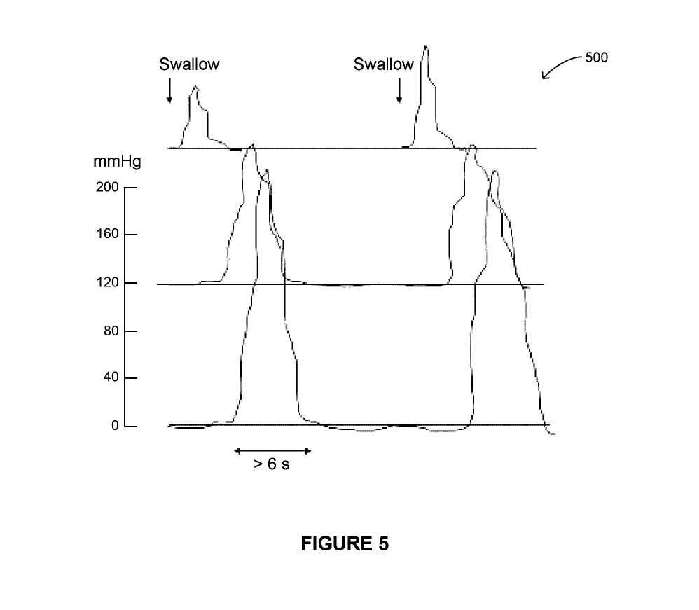

FIG. 5 shows an illustration of graphical read out regarding the pressure differential when a patient swallows during eating a meal. In this manner, the band bladder (152,153), catheter 120, port chamber 340, transducer 310 combination can be used as a physiologic sensor, recording the act of swallowing food as the patient initiates a meal. The volume of the meal and its size can cause corresponding deflections in the pressure waveforms. This enables the current invention to estimate the meal size. The frequency of meals is also recorded. This information is sent to the CPU or control unit 700 by means of hard wire or wireless communication.

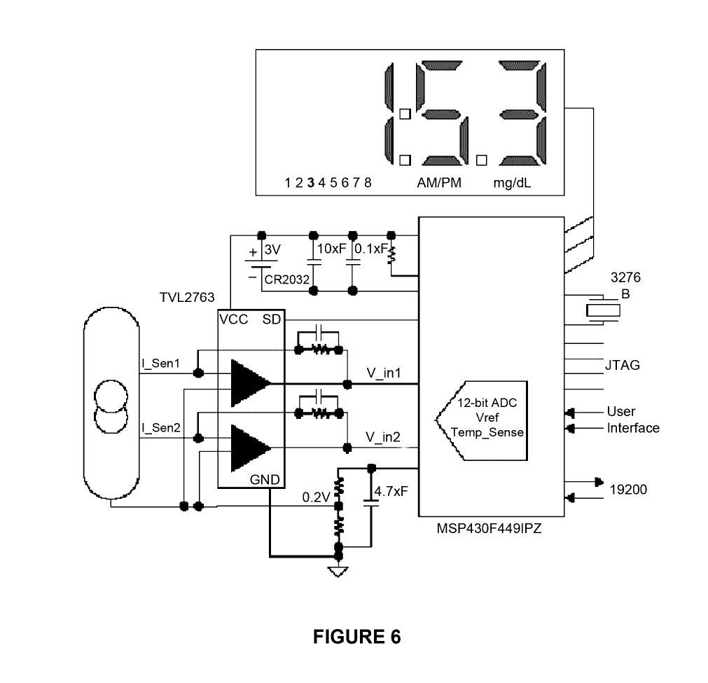

FIG. 6 shows a depiction of a glucose monitor. Depending on the embodiment the glucose monitor may be continuous or intermittent. The glucose monitor may be activated by the patient swallowing or drinking food. The MSA or Meal Sensing Apparatus functions in the presence of the glucose monitoring that may be implanted subcutaneously or known as a subcutaneous glucose monitor (SGM) device. The SGM device measures the glucose level before and after meal digestion. This information is transmitted to the CPU for use in its drug delivery algorithm.

The SGM obtains glucose levels from subcutaneous fluid. The timing of glucose measurement is an important feature of the present invention. Depending on the implementation, the command to obtain glucose levels from the SGM is initiated by the MSA. The moment that a meal is sensed by the MSA, the glucose level is obtained. Another glucose level may be obtained at 15, 30 and 60 minute intervals after eating. All the data collected by the SGM may be transmitted to a non-transitory computer readable medium either away from the patient or within the patient.

Depending on the embodiment, the SGM is implanted in communication with the MSA. However, it may also be a free standing external device, whether implanted or not implanted in the patient. In addition all the components of the system may be separate components or one integral component having the function of the above mentioned components of the gastric drug delivery device. The SGM communicates to the CPU either by wired or wireless communication.

Also included in the gastric drug delivery device, depending on the embodiment, is an EKG monitor. The EKG monitor may be implantable or not implantable and again may be an integral component or an individual component. The electrocardiographic monitor, whether included individually or with the other components of the present invention, monitors the heart rate of a patient. Heart rate is a sensitive marker of autonomic function. The digestive process, which is mainly a parasympathetic nervous system function, is associated with a slowing of the heart rate. This heart rate information is sent to the CPU by means of hard wire or wireless communication that further indicates whether or not the patient is eating and how much the patient has consumed.

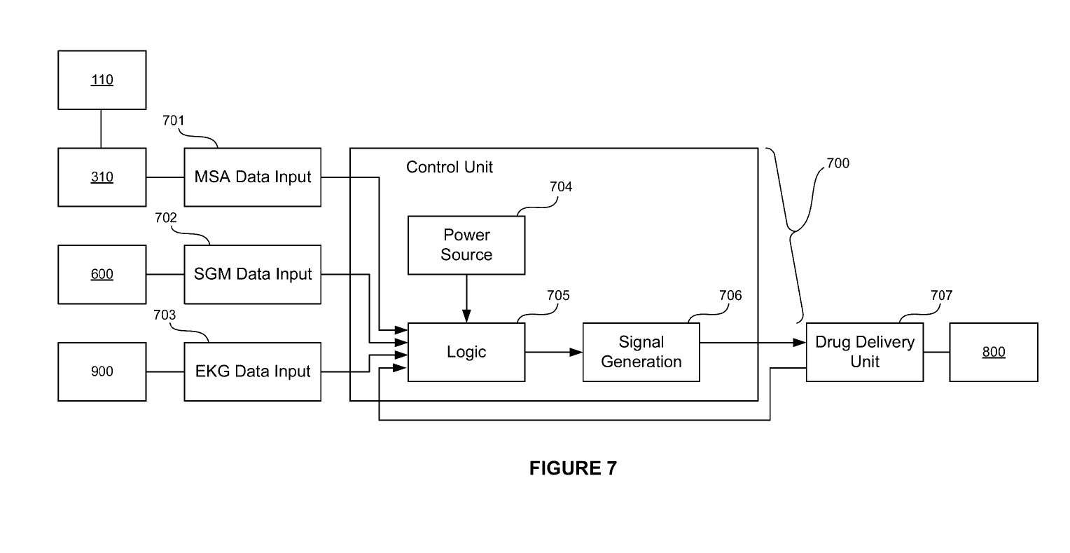

FIG. 7 illustrates a control unit 700 or CPU integrator system/controller. The control unit receives data inputs or signal 701, signal 702, and signal 703 from the MSA 110 and transducer 310, SGM 600 and EKG 900 units, respectively. These signals are processed through software 705 programmed in the control unit 700 that is powered by a power unit 704. Power unit 704 may be re-chargeable externally or internally depending on the implementation. A signal generation 706 in the control unit 700 is generated to respond to a variety of conditions including at least one of: the initiation of eating, the size of the meal, the number of swallowed food boluses, interval between meals, the change in glucose levels during digestion, the heart rate response during digestion and others, such as any combination listed.

The software performs a data analysis through an algorithm to determine whether or not the criteria for drug delivery have been met. If the result is an affirmative, a trigger signal 706 from the CPU sends a drug administration instruction to the drug delivery unit 707 having pump 800. The drug administration instruction is programmed to deliver a specific subcutaneous dose of an incretin drug or other active agent to the patient. The dosage is preprogrammed based on the algorithm.

The incretin drug dosage may be modified based upon the glucose reading from the CGM. The control unit 700 may receive patient input based upon hunger perception and knowledge of the meal's caloric content. The control unit 700 also has reporting capability regarding the band pressures, glucose levels and drug reservoir status. The control unit 700 may have wireless connectivity for interactions with a smart phone app or external computer. The incretin dose can also be suppressed by feedback from either the drug delivery system or the SGM. For example, if the patient received a dose within a lockout timeframe, the delivery would be suppressed. Similarly, if the patient's glucose level was dangerously low, the drug delivery would be suppressed.

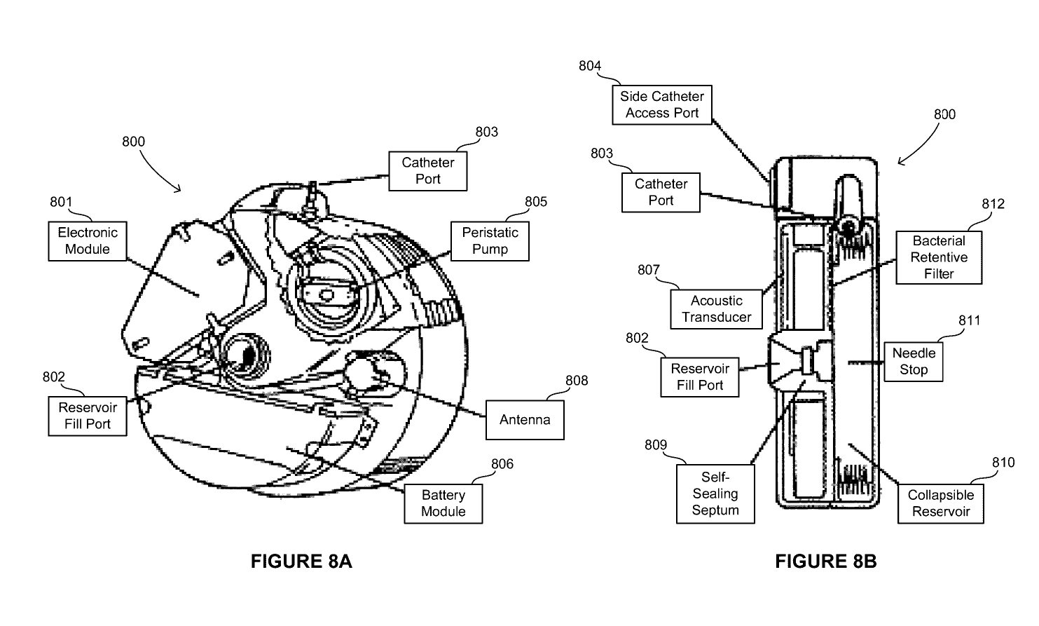

FIG. 8 illustrates the pump 800 of the drug delivery unit 707. The drug delivery unit (DDU) 707 is comprised of a subcutaneous access port (not shown), a refillable drug reservoir (not shown), a miniature infusion pump 800 and a subcutaneous catheter (not shown). Depending on the embodiment, the DDU is physically connected to the MSA port. However, it may be a separate, subcutaneously implanted port.

The DDU has a dual channel communication link with the CPU. The link can be either hard wired or wireless. The DDU receives instructions on the drug dose and timing from the CPU. The DDU transmits information on the drug delivery back to the CPU for confirmation. It also reports the status of the drug reservoir to the CPU so that an alert can be sent to the patient when a refill is needed. The DDU contains software to determine its battery charge and functional status to report to the CPU.

As shown in FIG. 8A and FIG. 8B, the pump 800 includes an electronic module 801 to control the pump. This module may or may not be replaced by the control unit 700. A reservoir fill port 802 is used to fill the pump with the active agent. This filling may be done through the subcutaneous access port described above. A catheter port 803 is used to connect to the subcutaneous catheter to deliver the active agent to the desired site or sites in the patient. The pump may also include a side catheter access port 804 as well. Depending on the embodiment, pump 800 may include a peristatic pump 805, battery module 806, acoustic transducer 807 and antenna 808 for wireless communication.

A self-sealing septum 809 in the reservoir fill port may also be included to avoid leakage of the active agent. Needle stop 811 may be included to avoid needle puncture into collapsible reservoir 810 that may hold the active agent. A bacterial retentive filter may also be utilized in device 800.

One example of how the process works or algorithm logic is utilized in the present invention is as follows. MSA detects pressure change in band bladder, indicating meal initiation. A Signal pattern is sent to the CPU. The CPU instructs SGM to measure glucose level. The CPU determines glucose level is above hypoglycemic parameters. The CPU records heart rate. The CPU determines heart rate slowing. The CPU records last dose delivered. The CPU determines dose time exceeds lockout time. CPU determines drug reservoir has adequate content. CPU determines drug delivery system is charged and functional. CPU instructs DDU to administer an initial dose of medication. CPU notes glucose excursion and heart rate changes. If glucose exceeds set parameters, CPU instructs DDU to administer a second dose of medication.

This invention is utilized for the purpose of diabetes resolution by combining gastric restrictive surgery with incretin drug delivery. It is demonstrated that gastric restriction by means of an inflatable gastric band can be combined with an incretin drug to induce a metabolic change leading to reversal of type II diabetes. The combination of gastric restriction and incretin therapy also contributes to weight loss.

By using the current invention, the meal sensing apparatus (MSA) acts as a trigger to the CPU. The CPU then instructs the drug delivery unit (DDU) to deliver the incretin drug at a physiologically appropriate time, mimicking the process of normal digestion. The incretin effect modulates further food intake and regulates insulin secretion. Together, these actions control glucose intake, improve insulin secretion and produce long term weight loss. The weight loss, in turn, improves insulin sensitivity, further enhancing diabetes resolution. Representative examples of drugs, e.g., agents that produce in incretin-like effect, that may be used in the present invention, e.g., disposed in and released from the DDU, are disclosed in U.S. Pat. No. 8,710,002, the contents of which are incorporated by reference herein in their entirety.

Other drugs that affect the gastrointestinal tract may be considered as embodiments of this invention. In particular, the invention can be applied to drugs which are meant to be given in a manner that links their effect to a particular phase of the digestive process. The digestive process begins with the initiation of meal consumption and specific digestive actions can be expected to occur at timed intervals thereafter.

One example of such an agent would be the use of anti-emetic drugs in chronic conditions associated with severe nausea and vomiting. Particular examples include diseases such as gastroparesis and upper gastrointestinal tract dysmotility disorders. Metoclopramide and granisetron are pharmaceuticals commonly prescribed to patients with these conditions. In this example, the invention's meal sensing apparatus would be used to trigger a subcutaneous injection of one or both of these agents for the purpose of controlling nausea and promoting proper antegrade gastric motility. The use of the invention permits the dose of medication(s) to occur at a physiologically appropriate time. This results in better symptom control for the patient.

Another example of such an agent would be octreotide in the management of chronic secretory diarrhea. Octreotide is used for this condition by means of a subcutaneous injection after each meal. In this example, the invention's meal sensing apparatus would be used to trigger a subcutaneous injection of octreotide in a manner timed to the meal initiation. This would allow a proper synchronization of the drug's effect with the appropriate stage of the digestive process. This results in better symptom control for the patient.

Non gastrointestinal drugs linked to measurable organ distension. Drugs that are utilized for other body systems may be optimized if they are administered in relation to a pathophysiologic state of distension. In this context, the current invention can be applied to any condition in which a globular organ becomes physically enlarged during illness. Such an occurrence would prompt the treatment with an agent that could reverse the distension or enlargement.

An example of such an agent would be in the treatment of Congestive Heart Failure with diuretics. When the heart enlarges due to inadequate inotropy, excess fluid accumulates in its chambers. Drugs which eliminate this fluid, such as diuretics, have been a mainstay of therapy for this condition. However, these agents are generally either administered after the condition has occurred or in anticipation of the problem. As a result, a higher dosage may be necessary to obtain the correct impact, exposing the patient to more adverse effects. Even worse, the drug may be administered too late to prevent further deterioration in cardiac function. The current invention could be utilized to provide a dose of diuretic at a more physiologically appropriate time.

In this example, the physiologic sensor would be stretchable mesh rather than the band bladder described in the gastrointestinal tract examples. The mesh would be implanted along the posterior wall of the left atrium. As the left atrium becomes engorged and swells, the degree of enlargement is detected by the mesh and a trigger signal sent to the CPU. The CPU interprets this signal and instructs the DDU to administer a subcutaneous dose of diuretic, such as furosemide.

Another example of such an agent would be in the treatment of urinary incontinence due to an overfilled bladder. When this condition is due to a weakening of the detrusor muscle, drugs such as oxybutynin have become a mainstay of therapy. However, these agents are generally either administered after the symptoms are severe or in anticipation of the problem. As a result, a higher dosage may be necessary to obtain the correct impact, exposing the patient to more adverse effects. The current invention could be utilized to provide a dose of medication at a more physiologically appropriate time. In this example, the physiologic sensor would be stretchable mesh rather than the band bladder described in the gastrointestinal tract examples. The mesh would be implanted along the posterior wall of the urinary bladder. As the bladder becomes engorged and swells, the degree of enlargement is detected by the mesh and a trigger signal sent to the CPU. The CPU interprets this signal and instructs the DDU to administer a subcutaneous dose of medication.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A gastric drug delivery device for improving diabetes patient care, comprising:
a meal sensor apparatus having a base; the base defining a chamber;
a pressure transducer disposed within the meal sensor apparatus, and the pressure transducer in fluid communication with the chamber;
a gastric band bladder in fluid communication with the chamber, and wherein deflection of the band bladder causes a pressure differential in the chamber that is readable by the transducer.

2. The gastric drug delivery device of claim 1, further including a control unit that receives information from the transducer and determines a volume food consumed based on corresponding waveforms of the pressure differential read by the pressure transducer.

3. The gastric drug delivery device of claim 2 wherein the control unit further includes a memory to record at least one of a frequency of meals, and a volume of meals.

4. The gastric drug delivery device of claim 2 wherein information from the transducer to the control unit is sent either by wireless connection or wire connection.

5. The gastric drug delivery device of claim 1, where the pressure differential is caused by a patient swallowing food or drinking a liquid.

6. The gastric drug delivery device of claim 2 further including a drug delivery system, the drug delivery system including a drug delivery pump that is activated by the information provided by the meal sensor apparatus.

7. The gastric drug delivery device of claim 6 further including a glucose monitor in communication with the control unit.

8. The gastric drug delivery device of claim 7 wherein the glucose monitor monitors glucose blood levels of the patient and wherein the monitoring is activated by the pressure differential.

9. The gastric drug delivery device of claim 2 further including an EKG monitor in communication with the control unit.

10. The gastric drug delivery device of claim 1 further including a CPU system integrator/controller connected to the meal sensing apparatus for controlling a drug delivery system.

11. The gastric drug delivery device of claim 10 wherein the CPU system determines the volume of food and frequency of meals a patient consumes.

12. The gastric drug delivery device of claim 11 wherein the CPU system further includes a non-transitory computer readable medium that records at least one of data of the volume of food and data of the frequency of meals a patient consumes.

13. The gastric drug delivery device of claim 12 wherein the data is wireless or hard wired transmitted to a computer storage facility apart from the patient.

14. The gastric drug delivery device of claim 12 wherein the data is stored in an implantable storage unit.

15. An implantable gastric drug delivery device for improving diabetes patient care, comprising:
an implantable meal sensor apparatus having a base; the base defining a chamber; a pressure transducer disposed within the meal sensor apparatus, and the pressure transducer in fluid communication with the chamber;
a gastric band bladder in fluid communication with the chamber, and wherein movement of the band bladder causes a pressure differential in the chamber that is readable by the transducer;
an implantable EKG monitor;
an implantable glucose monitor;
an implantable CPU system integrator/controller configured to receive information from the meal sensor apparatus, EKG monitor and glucose monitor and to determine the volume of food consumed; and
a drug delivery pump in communication with the implantable CPU system, the drug delivery pump for pumping an active agent at the command of the CPU system integrator/controller based on the data received by the CPU system in response to at least one of: an initiation of eating, size of the meal, number of swallowed food boluses, interval between meals, change in glucose levels during digestion, heart rate response during digestion and any combination thereof.

16. A process of treating of diabetes through a gastric drug delivery device, comprising:
reading a pressure differential by a meal sensing apparatus having a transducer disposed in a subcutaneous port, wherein the pressure differential is between a gastric band bladder connected to a catheter, and a chamber in the subcutaneous port, and wherein movement of the band bladder causes the pressure differential through the catheter and chamber that is readable by the transducer; and
initiating a drug delivery pump to deliver an active agent.

17. The process in claim 16 further including:
monitoring blood glucose levels, the monitoring being initiated when the transducer senses the pressure differential from the patient eating a meal.

18. The process in claim 17 further including monitoring the heart rate of a patient by an EKG monitor.

19. The process in claim 18, wherein the meal sensor apparatus, glucose monitor and EKG monitor are all implantable devices connected together.

20. The process in claim 16, further including recording in a non-transitory readable medium located in a control unit an act of swallowing food as a patient initiates a meal to determine at least one of: the frequency of the patient eating and a volume of meals.
